(19) United States
(12) Patent Application Publication
(10) Pub. No.: US 2005/0075626 A1
(43) Pub. Date: Apr. 7, 2005

(54) Multilayer tubular structure with application in catheter manufacturing

(76) Inventor: Andrea Venturelli, Roncadelle (IT)

Correspondence Address: SHOEMAKER AND MATTARE, LTD, 10 POST OFFICE ROAD - SUITE 110, SILVER SPRING, MD, 20910, US

(21) Appl. No.: 10/502,800

(22) PCT Filed: Feb. 17, 2003
(86) PCT No.: PCT/IT03/00086

(30) Foreign Application Priority Data
Feb. 26, 2002 (IT) BS2002U000024

(51) Int. Cl. A61M 25/00

(52) U.S. Cl. 604/524

(57) ABSTRACT

This invention concerns a multilayer tubular body for the manufacture of catheter devices, which comprises an outer layer having certain mechanical properties and weldability, an inner layer with lubricious characteristics and forming at least one longitudinal lumen, and one intermediate bonding layer between the outer and inner layers. The intermediate bonding layer is, in cross-section, interrupted and in the form of spaced apart circular sectors, and the inner layer has on the inside portions which alternate with the sectors in the intermediate layer.

### BACKGROUND OF THE INVENTION

This invention concerns catheters in general and refers in particular to an improved multilayer extruded tubing suitable for producing intravascular or similar catheters.

A trilayer coextruded tubular body for use in medical tubing and medical devices utilizing such tubing has already been proposed. A trilayer tubular body, used in a large number of devices for therapeutic and diagnostic purposes is described for example in U.S. Pat. No. 6,165,166.

This tubular body comprises an outer layer, an inner layer and an intermediate bonding layer between the outer and inner layers. The outer layer is made of a polymer, such as polyester, polyamide or a combination of both, having a first glass transition temperature. The inner layer, which forms at least one longitudinal lumen, is made up of a lubricious polymer, such as an olefinic or a fluorinate polymer, having a third glass transition temperature. The intermediate layer is made up of a functionalized polymer, such as olefinic modified with either anhydride or acrylate, having a second glass transition temperature.

The outer layer is to provide the resulting tubing with the characteristics of resistance, pushability, torsion, flexibility and weldability, whereas the inner lubricious layer enables the guide wire or any other means inserted into it to slide freely, and the intermediate layer stably bonds the otherwise incompatible and subject to detaining outer and inner layers.

However, the trilayers each have a uniform thickness along all the length of the tubular body and nothing in the above mentioned U.S. patent suggests that it could be otherwise.

In the catheters, the slippery aspect of the lumen or lumens formed by the inner layer is important, but also important if not even more important still are the mechanical characteristics they must have. It is therefore necessary for the coextruded structure of a tubular body for producing catheters and medical devices to really meet these requirements.

### SUMMARY OF THE INVENTION

It is in fact the objective of the present invention to make and provide a multilayer tubular body for catheter structures where a greater importance is given to the mechanical characteristics of resistance, torsion, flexibility and pushability through a specific combination of the coextruded layers and a different configuration of the outer layer.

This objective is achieved with a trilayer tubular body in which the outer layer has on the inside, in cross section, sectors which penetrate into the intermediate layer, in this way reducing the presence of the intermediate layer and locally increasing the thickness of the outer layer in order to increase the mechanical properties of the resulting tubular body.

### BRIEF DESCRIPTION OF THE DRAWINGS

Greater detail of the invention will become clear from the continuation of the present specification made with reference to the enclosed indicative and non-limiting drawings, in which:

FIG. 1 is an enlarged cross-section view of a trilayer tubular body; and

FIG. 2 is a view in perspective of a length of tubular body.

### DETAILED DESCRIPTION OF THE INVENTION

As shown, the tubular body includes an outer layer 11, an inner layer 12 and an intermediate layer 13. Said tubular body can be extruded, extruding the three layers together and choosing an appropriate polymer for each one. Each layer has a given thickness and the inner layer forms at least one longitudinal lumen 14.

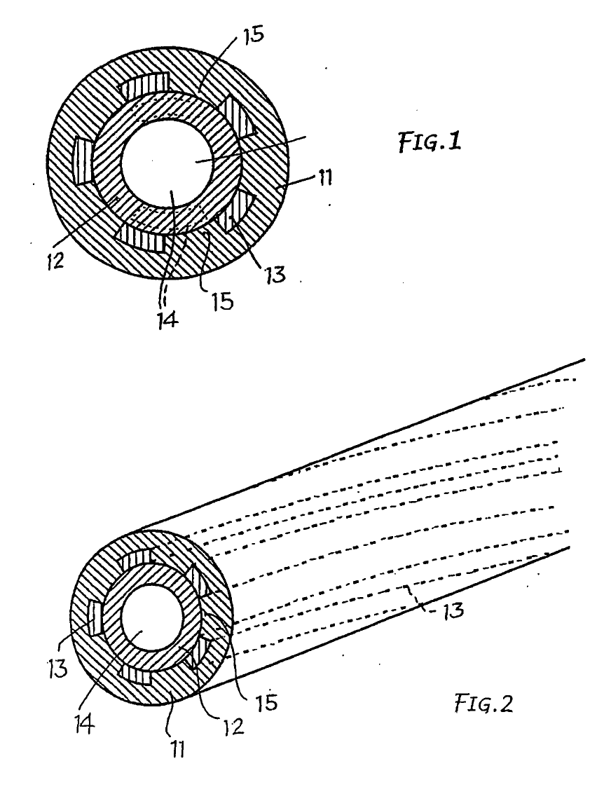

In particular, the outer layer has on the inside, in cross section—FIG. 1, protuberances or sectors 15, of the same or different in size, which penetrate into the thickness of the intermediate layer 13 which in this way is discontinuous and has the relevant joining sectors which alternate with the internal sectors 15 of said outer layer. The internal sectors 15 of the outer layer and in the same way the sectors formed by the intermediate layer 13 may be provided running lengthwise along the tubular body parallel to the axis of the latter or running helicoidally as shown in FIG. 2.

The outer layer 11 is committed to establishing the mechanical properties of the whole plus its weldability with other components of a catheter. The inner layer 12 ensures the smooth movement of the elements which are inserted into it. The presence of the intermediate layer 13, usually having mechanical properties inferior to those of the outer layer 11, according to this invention is limited, allowing an increase in the ratio and contribution of the outer layer 11 without jeopardizing, even if discontinuous and reduced to sectors, the bond between the outer and inner layers to avoid them detaching.

The result is, as said above, particular attention being paid to the mechanical characteristics of the tubular body and consequently to the catheter device o be made, without influencing the normal sizes and other functional aspects of the body itself.

1. A multilayer tubular body in particular for the manufacture of catheter devices, comprising an outer layer in a polymer with predefined mechanical and weldability properties, an inner layer in a polymer with lubricious characteristics and forming at least one longitudinal lumen, and an intermediate bonding layer between the outer and inner layers, wherein the intermediate bonding layer is, in cross section, in the form of circular sectors spaced between them, and the outer layer has on the inside parts which alternate with the sectors of the intermediate layer.

2. Multilayer tubular body according to claim 1, wherein the outer layer has internal protuberances extending longitudinally, the intermediate layer is discontinuous having spaced apart sectors, and the internal protuberances of outer layer are inserted into the gaps in the intermediate layer sectors.

3. Multilayer tubular body according to claims 1, wherein the outer layer has internal protuberances which alternate in turns with the gaps in the intermediate layer sectors, said internal protuberances and the intermediate layer sectors having the same thickness.

4. Multilayer tubular body according to claim 1, wherein the internal protuberances of the outer layer and the sectors forming the intermediate bonding layer run lengthways either parallel to or helicoidally to the axial of the body.
